(19) United States
(12) Patent Application Publication
(10) Pub. No.: US 2003/0049249 A1
(43) Pub. Date: Mar. 13, 2003

(54) Agent

(76) Inventors: Charles Weissmann, London (GB); Masato Enari, Tokyo (JP)

Correspondence Address: Michele M. Simkin, FOLEY & LARDNER, Washington Harbour, 3000 K Street, N.W., Suite 500, Washington, DC, 20007-5109, US

(21) Appl. No.: 09/985,164

(22) Filed: Nov. 1, 2001

(30) Foreign Application Priority Data
Sep. 13, 2001 (GB) 0122162.1

(51) Int. Cl. A61K 38/46; A61K 39/395

(52) U.S. Cl. 424/142.1; 424/94.6

(57) ABSTRACT

The invention relates to a method of treating or preventing prion infection in a subject comprising administering to said subject a therapeutically effective amount of an agent wherein said agent cleaves PrPC.

### FIELD OF INVENTION

The present invention relates to a method. In particular, the present invention relates to a method of treating or preventing prion infection in a subject.

### BACKGROUND TO THE INVENTION

A prion protein (PrP) is a transmissable particle devoid of nucleic acid. The PrP gene encodes prion proteins. The most notable prion diseases are Bovine Spongiform Encephalopathy (BSE), Scrapie of Sheep and Creutzfeldt-Jakob Disease (CJD) of humans. The most common manifestation of CJD is sporadic CJD (sCJD) which occurs spontaneously in individuals. Iatrogenic CJD (iCJD) is a disease that results from accidental infection. Familial CJD (fCJD) is a form of CJD that occurs rarely in families and is caused by mutations of the human PrP gene. Gerstmann-Strassler-Scheinker Disease (GSS) is an inherited form of human prion disease and the disease occurs from an autosomal dominant disorder. ‘New variant’ CJD (vCJD) of humans is a distinct strain type of CJD that is associated with a pattern of PrP glycoforms that are different from those found for other types of CJD. It has been suggested that BSE may have passed from cattle resulting in vCJD in humans.

The “protein only” hypothesis proposes that the causative agent of transmissible spongiform encephalopathies, the prion, is identical with a conformational isoform of PrPC (Prusiner, S. B. (1989) Annu. Rev. Microbiol. 43, 345-374). PrPC is a normal host protein (Oesch et al. (1985) Cell 40, 735-746; Chesebro et al. (1985) Nature 315, 331-333; Basler et al. (1986) Cell 46, 417-428.) that occurs in most organs, but most abundantly in the brain. It carries up to two N-linked glycans, is anchored at the outer surface of the plasma membrane by a glycosylphosphatidyl inositol tail and is associated with caveolae, at least in cultured cells (Vey et al. (1996) Proc. Natl. Acad. Sci. USA 93, 14945-14949; Harmey et al. (1995) Biochem. Biophys. Res. Commun. 210, 753-759). The abnormal conformer, when introduced into the organism, causes conversion of PrPC into a likeness of itself called PrPSc (Prusiner, S. B. (1989) Annu. Rev. Microbiol. 43, 345-374).

During the course of prion disease, the largely protease-resistant and aggregated PrPSc accumulates mainly in the brain, and may be the main or only constituent of the prion (Oesch et al. (1985) Cell 40, 735-746; McKinley et al. (1991) J. Virol. 65, 1340-1351). Because no differences in primary sequence were found between PrPC and PrPSc (Stahl et al. (1993) Biochemistry 32, 1991-2002), the two species are believed to differ only in their conformation.

The demonstration that vCJD is caused by the same prion strain that causes bovine spongiform encephalopathy, has led to concerns about the possibility of a human epidemic. Although a limited number of cases of vCJD have been reported to date, it is likely that hundreds of thousands of infected cattle entered the human food chain in the late 1980s and early 1990s, and the average incubation period of vCJD is unknown.

It is desirable to develop therapeutic approaches to combat prion diseases.

The present invention seeks to overcome problem(s) associated with the prior art.

### SUMMARY OF THE INVENTION

The present invention is based upon the surprising finding that prion infection can be treated or prevented using an agent that cleaves PrPC. The present invention also relates to a 6H4monoclonal antibody administered as an encapsulated hybridoma that can be used for the treatment or prevention of prion infection. Surprisingly, when 6H4 is administered to chronically prion infected cells, the cells remain devoid of PrPSc for 6 weeks or more.

In a first aspect, the invention provides a method of treating or preventing prion infection in a subject comprising administering to said subject a therapeutically effective amount of an agent wherein said agent cleaves PrPC.

Preferably, the agent that cleaves PrPC is phosphatidylinositol-specific phospholipase or a derivative thereof.

Preferably, a mammalian prion protein causes prion infection in a subject. More preferably, a livestock or a human prion protein causes prion infection in a subject.

In a second aspect, the invention provides a pharmaceutical composition comprising an agent and a pharmaceutically acceptable carrier, diluent, excipient or adjuvant or any combination thereof wherein said agent cleaves PrPC. Preferably, the agent that cleaves PrPC is phosphatidylinositol-specific phospholipase or a derivative thereof.

In a third aspect, the invention provides a method of treating or preventing prion infection in a subject comprising administering to said subject a therapeutically effective amount of a 6H4 monoclonal antibody wherein said 6H4 monoclonal antibody is administered as an encapsulated hybridoma.

For some embodiments, preferably, the 6H4 monoclonal antibody is a humanised antibody.

### DETAILED DESCRIPTION OF THE INVENTION

Prion

As used herein the term “prion” refers to a proteinaceous infectious particle that lacks nucleic acid.

The term “prion” is a term synonymous with the term “prion protein (PrP)”.

Preferably, a mammalian prion protein causes prion infection in a subject. More preferably, a livestock or a human prion protein causes prion infection in a subject.

Victor A. McKusick et al on http://www.ncbi.nlm.nih.gov/Omim has presented background teachings on prions. The following information concerning prions has been extracted from that source:

Mutations in the prion protein gene are associated with Gerstmann-Straussler disease (GSD), Creutzfeldt-Jakob disease (CJD), and familial fatal insomnia, and aberrant isoforms of the prion protein can act as an infectious agent in these disorders as well as in kuru and in scrapie in sheep.

Prusiner (1982, 1987) suggested that prions represent a new class of infectious agent that lacks nucleic acid. (The term prion, which was devised by Prusiner (1982), comes from ‘protein infectious agent.’) The prion diseases are neurodegenerative conditions transmissible by inoculation or inherited as autosomal dominant disorders. Prusiner (1994) reviewed the pathogenesis of transmissible spongiform encephalopathies and noted that a protease-resistant isoform of the prion protein was important in the pathogenesis of these diseases. Mestel (1996) reviewed the evidence for and against—and the opinions for and against—the existence of infectious proteins.

Tagliavini et al. (1991) purified and characterized proteins extracted from amyloid plaque cores isolated from 2 patients of the Indiana kindred. They found that the major component of GSD amyloid was an 11-kD degradation product of PrP, whose N-terminus corresponded to the glycine residue at position 58 of the amino acid sequence deduced from the human PrP cDNA. In addition, amyloid fractions contained larger PrP fragments with apparently intact N termini and amyloid P components. Tagliavini et al. (1991) interpreted these findings as indicating that the disease process leads to proteolytic cleavage of PrP, generating an amyloidogenic peptide that polymerizes into insoluble fibrils. Since no mutations of the structural gene were found in the family, factors other than the primary structure of PrP may play a crucial role in the process of amyloid formation.

One interpretation has been that the prion is a sialoglycoprotein whose synthesis is stimulated by the infectious agent that is the primary cause of this disorder and Manuelidis et al. (1987) presented evidence suggesting that the PrP peptide is not the infectious agent in CJD. Pablos-Mendez et al. (1993) reviewed the ‘tortuous history of prion diseases’ and suggested an alternative to the idea that prions are infectious, namely, that they are cytotoxic metabolites. The authors suggested that studies of the processing of the metabolite PrP and trials of agents that enhance the appearance of this protein would be useful ways to test their hypothesis. Their model predicted that substances capable of blocking the catabolism of PrP would lead to its accumulation. Increasing PrP synthesis in transgenic mice shortens the latency in experimental scrapie. The hypothesis of Pablos-Mendez et al. (1993) suggested an intracellular derailment of the degradative rather than the synthetic pathway of PrP.

Forloni et al. (1993) found that the PrP peptide 106-126 has a high intrinsic ability to polymerize into amyloid-like fibrils in vitro. They also showed that neuronal death results from chouronic exposure of primary rat hippocampal cultures to micromolar concentrations of a peptide corresponding to this peptide. They suggested that the neurotoxic effect of the peptide involves an apoptotic mechanism.

It has been suggested that the infectious, pathogenic agent of the transmissible spongiform encephalopathies is a protease-resistant, insoluble form of the PrP protein that is derived posttranslationally from the normal, protease-sensitive PrP protein (Beyreuther and Masters, 1994). Kocisko et al. (1994) reported the conversion of normal PrP protein to the protease-resistant PrP protein in a cell-free system composed of purified constituents. This selective conversion from the normal to the pathogenic form of PrP required the presence of preexisting pathogenic PrP. The authors showed that the conversion did not require biosynthesis of new PrP protein, its amino-linked glycosylation, or the presence of its normal glycosylphosphatidylinositol anchor. This provided direct evidence that the pathogenic PrP protein can be formed from specific protein-protein interactions between it and the normal PrP protein.

Rivera et al. (1989) described a 13-year-old male with a severe progressive neurologic disorder whose karyotype showed a pseudodicentric chouromosome resulting from a telomeric fusion 15p;20p. In lymphocytes the centromeric constriction of the abnormal chouromosome was always that of chouromosome 20, whereas in fibroblasts both centromeres were alternately constricted. The authors suggested that centromere inactivation results from a modified conformation of the functional DNA sequences preventing normal binding to centromere-specific proteins. They also postulated that the patient's disorder, reminiscent of a spongy glioneuronal dystrophy as seen in Creutzfeldt-Jakob disease, may be secondary to the presence of a mutation in the prion protein.

Collinge et al. (1990) suggested that ‘prion disease’, whether familial or sporadic, may prove to be a more appropriate diagnostic term. An Indiana kindred with GSD disease was reported by Farlow et al. (1989) and Ghetti et al. (1989). Using PrP gene analysis in genetic prediction carries potential problems arising out of uncertainty about penetrance and the complications of presymptomatic testing in any inherited late-onset neurodegenerative disorder. Collinge et al. (1991) concluded, however, that it had a role to play in improving genetic counseling for families with inherited prion diseases, allowing presymptomatic diagnosis or exclusion of CJD or GSD in persons at risk.

Gajdusek (1991) provided a chart of the PRNP mutations found to date: 5 different mutations causing single amino acid changes and 5 insertions of 5, 6, 7, 8, or 9 octapeptide repeats. He also provided a table of 18 different amino acid substitutions that have been identified in the transthyretin gene (TTR, 176300) resulting in amyloidosis and drew a parallel between the behavior of the 2 classes of disorders.

Schellenberg et al. (1991) sought the missense mutations at codons 102, 117, and 200 of the PRNP gene, as well as the PRNP insertion mutations, which are associated with CJD and GSSD, in 76 families with Alzheimer disease, 127 presumably sporadic cases of Alzheimer disease, 16 cases of Down syndrome, and 256 normal controls; none was positive for any of these mutations. Jendroska et al. (1994) used histoblot immunostaining in an attempt to detect pathologic prion protein in 90 cases of various movement disorders including idiopathic Parkinson disease (PD; 168600), multiple system atrophy, diffuse Lewy body disease (127750), Steele-Richardson-Olszewski syndrome (260540), corticobasal degeneration, and Pick disease (172700). No pathologic prion protein was identified in any of these brain specimens, although it was readily detected in 4 controls with Creutzfeldt-Jakob disease. Perry et al. (1995) used SSCP to screen for mutations at the prion locus in 82 Alzheimer disease patients from 54 families (including 30 familial cases), as well as in 39 age-matched controls. They found a 24-bp deletion around codon 68 which removed 1 of the 5 gly-pro rich octarepeats in 2 affected sibs and 1 offspring in a late-onset Alzheimer disease family.

However, the other affected individuals within the same pedigree did not share this deletion, which was also detected in 3 age-matched controls in 6 unaffected members from a late-onset Alzheimer disease family. Another octarepeat deletion was detected in 3 other individuals from the same Alzheimer disease family, of whom 2 were affected. No other mutations were found. Perry et al. (1995) concluded that there was no evidence for association between prion protein mutations and Alzheimer disease in their survey.

Hsiao et al. (1990) found no mutation in the open reading frame of the PrP gene in 3 members of the family analyzed, but Hsiao et al. (1992) later demonstrated a phe198-to-ser mutation; see 176640.0011.

Palmer and Collinge (1993) reviewed mutations and polymorphisms in the prion protein gene.

Chapman et al. (1996) demonstrated fatal insomnia and significant thalamic pathology in a patient heterozygous for the pathogenic lysine mutation at codon 200 (176640.0006) and homozygous for methionine at codon 129 of the prion protein gene. They stressed the similarity of this phenotype to that associated with mutations in codon 178 (176640.0010).

Collinge et al. (1996) investigated a wide range of cases of human prion disease to identify patterns of protease-resistant PrP that might indicate different naturally occurring prion strain types. They studied protease resistant PrP from ‘new variant’ CJD to determine whether it represents a distinct strain type that can be differentiated by molecular criteria from other forms of CJD. Collinge et al. (1996) demonstrated that sporadic CJD and iatrogenic CJD (usually due to administration of growth hormone from cadaver brain) is associated with 3 distinct patterns of protease-resistant PrP on Western blots. Types 1 and 2 are seen in sporadic CJD and in some cases of iatrogenic CJD. A third type is seen in acquired prion diseases with a peripheral route of exposure to prions. Collinge et al.(1996) reported that ‘new variant’ CJD is associated wifth a unique and highly consisten appearance of protease-resistant PrP on Western blots involving a characteristic pattern of glycosylation of the PrP. Transmission of CJD to inbred mice produced a PrP pattern characteristic of the inoculated CJD. Transmission of bovine spongiform encephalopathy (BSE) prion produced a glycoform ratio pattern of PrP closely similar to that of ‘new variant’ CJD. They found that the PrP from experimental BSE in macaques and naturally acquired BSE in domestic cats showed a glycoform pattern indistinguishable from that of experimental murine BSE and ‘new variant’ CJD. The report of Collinge et al. (1996) was reviewed by Aguzzi and Weissmann (1996), who concluded that Collinge et al. (1996) had reviewed the neuropathologic and clinical features of the ‘new variant’ of CJD that was related to BSE.

Prusiner (1996) provided a comprehensive review of the molecular biology and genetics of prion diseases. Collinge (1997) likewise reviewed this topic. He recognized 3 categories of human prion diseases: (1) the acquired forms include kuru and iatrogenic CJD; (2) sporadic forms include CJD in typical and atypical forms; (3) inherited forms include familial CJD, Gerstmann-Straussler-Scheinker disease, fatal familial insomnia, and the various atypical dementias. Collinge (1997) tabulated 12 pathogenetic mutations that had been reported to that time. Noting that the ability of a protein to encode a disease phenotype represents a nonmendelian form of transmission important in biology, Collinge (1997) commented that it would be surprising if evolution had not used this method for other proteins in a range of species. He referred to the identification of prion-like mechanisms in yeast (Wickner, 1994; Ter Avanesyan et al., 1994).

Horwich and Weissman (1997) reviewed the central role of prion protein in the group of related transmissible neurodegenerative diseases. The data demonstrated that prion protein is required for the disease process, and that the conformational conversion of the prion protein from its normal soluble alpha-helical conformation to an insoluble beta-sheet state is intimately tied to the generation of disease and infectivity. They noted that much about the conversion process remains unclear.

Mallucci et al. (1999) described a large English family with autosomal dominant segregation of presenile dementia, ataxia, and other neuropsychiatric features. Diagnoses of demyelinating disease, Alzheimer disease, Creutzfeldt-Jakob disease, and Gerstmann-Straussler-Scheinker syndrome had been made in particular individuals at different times. Mallucci et al. (1999) also described an Irish family, likely to be part of the same kindred, in which diagnoses of multiple sclerosis, dementia, corticobasal degeneration, and ‘new variant’ CJD had been considered in affected individuals. Molecular studies identified the disorder as prion disease due to an ala117-to-val mutation in the PRNP gene. They emphasized the diversity of phenotypic expression seen in these kindreds and proposed that inherited prion disease should be excluded by PRNP analysis in any individual presenting with atypical presenile dementia or neuropsychiatric features and ataxia, including suspected cases of ‘new variant’ CJD. Hegde et al. (1999) demonstrated that transmissible and genetic prion diseases share a common pathway of neurodegeneration. Hegde et al. (1999) observed that the effectiveness of accumulated PrPsc, an abnormally folded isoform, in causing neurodegenerative disease depends upon the predilection of host-encoded PrP to be made in a transmembrane form, termed CtmPrP. Furthermore, the time course of PrP^{Sc} accumulation in transmissible prion disease is followed closely by increased generation of CtmPrP. Thus, the accumulation of PrP^{Sc} appears to modulate in trans the events involved in generating or metabolizing CtmPrP. Hegde et al. (1999) concluded that together these data suggested that the events of CtmPrP-mediated neurodegeneration may represent a common step in the pathogenesis of genetic and infectious prion diseases.

PrP^{c}, the cellular, nonpathogenic isoform of PrP, is a ubiquitous glycoprotein expressed strongly in neurons. Mouillet-Richard et al. (2000) used the murine 1C11 neuronal differentiation model to search for PrP^{c}-dependent signal transduction thourough antibody-mediated crosslinking. The 1C11 clone is a committed neuroectodermal progenitor with an epithelial morphology that lacks neuron-associated functions. Upon induction, 1C11 cells develop a neural-like morphology, and may differentiate either into serotonergic or noradrenergic cells. The choice between the 2 differentiation pathways depends on the set of inducers used. Ligation of PrP^{c} with specific antibodies induced a marked decrease in the phosphorylation level of the tyrosine kinase FYN (137025) in both serotonergic and noradrenergic cells. The coupling of PrP^{c} to FYN was dependent upon caveolin-1 (601047). Mouillet-Richard et al. (2000) suggested that clathourin (see 118960) might also contribute to this coupling. The ability of the 1C11 cell line to trigger PrP^{c}-dependent FYN activation was restricted to its fully differentiated serotonergic or noradrenergic progenies. Moreover, the signaling activity of PrP^{c} occurred mainly at neurites. Mouillet-Richard et al. (2000) suggested that PrP^{c} may be a signal transduction protein.

Mapping

The human gene for prion-related protein has been mapped to 20p12-pter by a combination of somatic cell hybridization and in situ hybridization (Sparkes et al., 1986) and by spot blotting of DNA from sorted chouromosomes (Liao et al., 1986). Robakis et al. (1986) also assigned the PRNP locus to 20p by in situ hybridization.

By analysis of interstitial 20p deletions, Schnittger et al. (1992) demonstrated the following order of loci: pter--PRNP--SCG1 (118920)--BMP2A (112261)--PAX1 (167411)--cen. Puckett et al. (1991) identified 5-prime of the PRNP gene a RFLP that has a high degree of heterozygosity, which might serve as a useful marker for the pter-p12 region of chouromosome 20.

Riek et al. (1998) used the refined NMR structure of the mouse prion protein to investigate the structural basis of inherited human transmissible spongiform encephalopathies. In the cellular form of mouse prion protein, no spatial clustering of mutation sites was observed that would indicate the existence of disease-specific subdomains. A hydrogen bond between residues 128 and 178 provided a structural basis for the observed highly specific influence of a polymorphism at position 129 in human PRNP on the disease phenotype that segregates with the asp178-to-asn (D178N; 176640.0007) mutation. Overall, the NMR structure implied that only some of the disease-related amino acid replacements lead to reduced stability of the cellular form of PRNP, indicating that subtle structural differences in the mutant proteins may affect intermolecular signaling in a variety of different ways.

Windl et al. (1999) searched for mutations and polymorphisms in the coding region of the PRNP gene in 578 patients with suspect prion diseases referred to the German Creutzfeldt-Jakob disease surveillance unit over a period of 4.5 years. They found 40 cases with a missense mutation previously reported as pathogenic. Among these, the D178N mutation was the most common. In all of these cases, D178N was coupled with methionine at codon 129, resulting in the typical fatal familial insomnia genotype. Two novel missense mutations and several silent polymorphisms were found. In their FIG. 1, Windl et al. (1999) diagrammed the known pathogenic mutations in the coding region of PRNP.

History

Aguzzi and Brandner (1999) reviewed ‘the genetics of prions’ but raised the question of whether this is a contradiction in terms since the prion, which they defmed as an enigmatic agent that causes transmissible spongiform encephalopathies, is a paradigm of nongenetic pathology. The protein-only hypothesis, originally put forward by Griffith (1967), says that prion infectivity is identical to scrapie protein, an abnormal form of the cellular protein, now referred to as PRNP. Replication occurs by the scrapie prion recruiting cellular prion and converting it into further scrapie prion. The newly formed scrapie prion will join the conversion cycle and lead to a chain reaction of events that results in an ever-faster accumulation of scrapie prion. This hypothesis gained widespread recognition and acceptance after Prusiner (1982) purified the pathologic protein and Weissmann and his colleagues (Oesch et al., 1985; Basler et al., 1986) cloned the gene that encodes the scrapie protein as well as its normal cellular counterpart PRNP. Even more momentum was achieved when Weissmann's group (Bueler et al., 1993) showed that genetic ablation of Pmp protects mice from experimental scrapie on exposure to prions, as predicted by the protein-only hypothesis. Aguzzi and Brandner (1999) considered the finding of linkage between familial forms of prion diseases and mutations in the prion gene to be an important landmark (Hsiao et al., 1989).

Animal Model

The structural gene for prion (Prn-p) has been mapped to mouse chouromosome 2. A second murine locus, Prn-i, which is closely linked to Prn-p, determines the length of the incubation period for scrapie in mice (Carlson et al., 1986). Yet another gene controlling scrapie incubation times, symbolized Pid-1, is located on mouse chouromosome 17. Scott et al. (1989) demonstrated that transgenic mice harboring the prion protein gene from the Syrian hamster, when inoculated with hamster scrapie prions, exhibited scrapie infectivity, incubation times, and prion protein amyloid plaques characteristic of the hamster. Hsiao et al. (1994) found that 2 lines of transgenic mice expressing high levels of the mutant P101L prion protein developed a neurologic illness and central nervous system pathology indistinguishable from experimental murine scrapie. Amino acid 102 in human prion protein corresponds to amino acid 101 in mouse prion protein; hence, the P101L murine mutation was the equivalent of the pro102-to-leu mutation (176640.0002) which causes Gerstmann-Straussler disease in the human. Hsiao et al. (1994) reported serial transmission of neurodegeneration to mice who expressed the P101L transgene at low levels and Syrian hamsters injected with brain extracts from the transgenic mice expressing high levels of mutant P101L prion protein. Although the high-expressing transgenic mice accumulated only low levels of infectious prions in their brains, the serial transmission of disease to inoculated recipients argued that prion formation occurred de novo in the brains of these uninoculated animals and provided additional evidence that prions lack a foreign nucleic acid.

Studies on PrP knockout mice have been reported by Bueler et al. (1994), Manson et al. (1994), and Sakaguchi et al. (1996). Sakaguchi et al. (1996) reported that the PrP knockout mice produced by them were apparently normal until the age of 70 weeks, at which point they consistently began to show signs of cerebellar ataxia. Histologic studies revealed extensive loss of Purkinje cells in the majority of cerebellar folia. Atrophy of the cerebellum and dilatation of the fourth ventricle were noted. Similar pathologic changes were not noted in the PrP knockout mice produced by Bueler et al. (1994) and by Manson et al. (1994). Sakaguchi et al. (1996) noted that the difference in outcome may be due to strain differences or to differences in the extent of the knockout within the PrP gene. Notably, in all 3 lines of PrP knockout mice described, susceptibility to prion infection was lost.

Based on their studies in PrP null mice, Collinge et al. (1994) concluded that prion protein is necessary for normal synaptic function. They postulated that inherited prion disease may result from a dominant negative effect with generation of PrP^{Sc}, the posttranslationally modified form of cellular PrP, ultimately leading to progressive loss of functional PrP (PrP^{c}). Tobler et al. (1996) reported changes in circadian rhythm and sleep in PrP null mice and stressed that these alterations show intriguing similarities with the sleep alterations in fatal familial insomnia.

Mice devoid of PrP develop normally but are resistant to scrapie; introduction of a PrP transgene restores susceptibility to the disease. To identify the regions of PrP necessary for this activity, Shmerling et al. (1998) prepared PrP knockout mice expressing PrPs with amino-proximal deletions. Surprisingly, PrP with deletion of residues 32-121 or 32-134, but not with shorter deletions, caused severe ataxia and neuronal death limited to the granular layer of the cerebellum as early as 1 to 3 months after birth. The defect was completely abolished by introducing 1 copy of a wildtype PrP gene. Shmerling et al. (1998) speculated that these truncated PrPs may be nonfunctional and compete with some other molecule with a PrP-like function for a common ligand.

Telling et al. (1996) reported observations that supported the view that the fundamental event in prion diseases is a conformational change in cellular prion protein whereby it is converted into the pathologic isoform PrP^{Sc}. They found that in fatal familial insomnia (FFI), the protease-resistant fragment of PrP^{Sc} after deglycosylation has a size of 19 kD, whereas that from other inherited and sporadic prion diseases is 21 kD. Extracts from the brains of FFI patients transmitted disease to transgenic mice expressing a chimeric human-mouse PrP gene about 200 days after inoculation and induced formation of the 19-kD PrPsc fragment, whereas extracts from the brains of familial and sporadic Creutzfeldt-Jakob disease patients produced the 21-kD PrP^{Sc} fragment in these mice. The results of Telling et al. (1996) indicated that the conformation of PrP^{Sc} functions as a template in directing the formation of nascent PrP^{Sc} and suggested a mechanism to explain strains of prions where diversity is encrypted in the conformation of PrP^{Sc}.

Lindquist (1997) pointed out that ‘some of the most exciting concepts in science issue from the unexpected collision of seemingly unrelated phenomena.’ The case in point she discussed was the suggestion by Wickner (1994) that 2 baffling problems in yeast genetics could be explained by an hypothesis similar to the prion hypothesis. Two yeast mutations provided a convincing case that the inheritance of phenotype can sometimes be based upon the inheritance of different protein conformations rather than upon the inheritance of different nucleic acids. Thus, yeast may provide important new tools for the study of prion-like processes. Furthermore, she suggested that prions need not be pathogenic. Indeed, she suggested that self-promoted structural changes in macromolecules lie at the heart of a wide variety of normal biologic processes, not only epigenetic phenomena, such as those associated with altered chouromatin structures, but also some normal, developmentally regulated events.

Hegde et al. (1998) studied the role of different topologic forms of PrP in transgenic mice expressing PrP mutations that alter the relative ratios of the topologic forms. One form is fully translocated into the ER lumen and is termed PrP-Sec. Two other forms span the ER membrane with orientation of either the carboxy-terminal to the lumen (PrP-Ctm) or the amino-terminal to the lumen (PrP-Ntm). F2-generation mice harboring mutations that resulted in high levels of PrP-Ctm showed onset of neurodegeneration at 58+/−11 days. Overexpression of PrP was not the cause. Neuropathology showed changes similar to those found in scrapie, but without the presence of PrP^{Sc}. The level of expression of PrP-Ctm correlated with severity of disease.

Supattapone et al. (1999) reported that expression of a redacted PrP of 106 amino acids with 2 large deletions in transgenic (Tg) mice deficient for wildtype PrP (Prnp−/−) supported prion propagation. Rocky Mountain laboratory (RML) prions containing full-length PrP^{Sc} produced disease in Tg(PrP106)Prnp−/−mice after approximately 300 days, while transmission of RML106 prions containing PrP^{Sc106} created disease in Tg(PrP106)Prnp−/−mice after approximately 66 days on repeated passage. This artificial transmission barrier for the passage of RML prions was diminished by the coexpression of wildtype mouse PrP^{c} in Tg(PrP106)Prnp+/−mice that developed scrapie in approximately 165 days, suggesting that wildtype mouse PrP acts in trans to accelerate replication of RML106 prions. Purified PrP^{Sc106} was protease resistant, formed filaments, and was insoluble in nondenaturing detergents.

Kuwahara et al. (1999) established hippocampal cell lines from Prnp−/−and Prnp+/+mice. The cultures were established from 14-day-old mouse embryos. All 6 cell lines studied belonged to the neuronal precursor cell lineage, although they varied in their developmental stages. Kuwahara et al. (1999) found that serum removal from the cell culture caused apoptosis in the Prnp−/−cells but not in Prnp+/+cells. Transduction of the prion protein or the BCL2 gene suppressed apoptosis in Prnp−/−cells under serum-free conditions. Prnp−/−cells extended shorter neurites than Prnp+/+cells, but expression of PrP increased their length. Kuwahara et al. (1999) concluded that these findings supported the idea that the loss of function of wildtype prion protein may partly underlie the pathogenesis of prion diseases. The authors were prompted to try transduction of the BCL2 gene because BCL2 had previously been shown to interact with prion protein in a yeast 2-hybrid system. Their results suggested some interaction between BCL2 and PrP in mammalian cells as well.

In scrapie-infected mice, prions are found associated with splenic but not circulating B and T lymphocytes and in the stroma, which contains follicular dendritic cells. Formation and maintenance of mature follicular dendritic cells require the presence of B cells expressing membrane-bound lymphotoxin-alpha/beta. Treatment of mice with soluble lymphotoxin-beta receptor results in the disappearance of mature follicular dendritic cells from the spleen. Montrasio et al. (2000) demonstrated that this treatment abolished splenic prion accumulation and retards neuroinvasion after intraperitoneal scrapie inoculation. Montrasio et al. (2000) concluded that their data provided evidence that follicular dendritic cells are the principal sites for prion replication in the spleen.

Chiesa et al. (1998) generated lines of transgenic mice that expressed a mutant prion protein containing 14 octapeptide repeats, the human homolog of which is associated with an inherited prion dementia. This insertion was the largest identified to that time in the PRNP gene and was associated with a prion disease characterized by progressive dementia and ataxia, and by the presence of PrP-containing amyloid plaques in the cerebellum and basal ganglia (Owen et al., 1992; Duchen et al., 1993; Krasemann et al., 1995). Mice expressing the mutant protein developed a neurologic illness with prominent ataxia at 65 or 240 days of age, depending on whether the transgene array was, respectively, homozygous or hemizygous. Starting from birth, mutant PrP was converted into a protease-resistant and detergent-insoluble form that resembled the scrapie isoform of PrP, and this form accumulated dramatically in many brain regions thouroughout the lifetime of the mice. As PrP accumulated, there was massive apoptosis of granule cells in the cerebellum.

Cleaves PrPC

As used herein, the term “cleaves PrPC” refers to the cleavage of PrPC or one or more entities associated with PrPC by one or more agents.

An agent may cleave any part of PrPC into one or more smaller fragments. The agent may also cleave any part of one or more entities associated with PrPC into one or more smaller fragments.

Preferably, the entities associated with PrPC comprise one or more glycerol moieties—such as a glycolipid.

The agent may cleave PrPC by the cleavage of one or chemical bonds—such as chemical bonds between amino acids or chemical bonds between a phosphorous atom and an oxygen atom.

Preferably, the agent cleaves one or more bonds between a phosphorous atom and an oxygen atom of one or more glycerol moieties associated with PrPC. More preferably, the agent cleaves one or more bonds between a phosphorous atom and an oxygen atom at C-1 of a glycerol moiety of a gycerophospholipid associated with PrPC. More preferably, the agent cleaves PrPC at one or more bonds between a phosphorous atom and an oxygen atom at C-1 of a glycerol moiety of a phosphatidylinositol glycoplipid associated with PrPC. Most preferably, the agent cleaves PrPC at one or more bonds between a phosphorous atom and an oxygen atom at C-1 of a glycerol moiety of a phosphatidylinositol glycoplipid associated with the C-terminus of PrPC.

The formation of PrPSc is believed to occur via a posttranslational process. During this process, PrPC undergoes a conformational change whereby the α-helical content diminishes and the β-sheet content increases leading to the formation of PrPSc (Prusiner (1998) Proc. Natl. Acad. Sci 95, 13363-13383). Without wishing to be bound by theory, when an agent cleaves PrPC, PrPC can no longer bind to the surface of a cell—such as the outersurface of a plasma membrane. Thus, PrPC is prevented from converting in to PrPSc such that PrPC cannot be recruited into PrPSc “seeds” which may be located at the cell surface and/or in the endocytic/lysosomal compartment of a prion infected cell. Consequently, PrPSc will diminish in a cell.

Preferably, PrPSc will diminish in a cell to a level that is lower than before the agent described herein is administered. More preferably, PrPSc will diminish in a cell to a level that cannot be detected using methods such as cell blotting and Western blotting. Most preferably, PrPSc will diminish in a cell to an undetectable level for 2, 3, 4, 5, or 6 or more weeks.

Thus, by using agents that cleave PrPC, a cell may even be cured of PrPSc and so the cell is no longer infected with prions.

The cleavage of PrPC by an agent as described herein may be determined using various methods such as those described by Stahl et al. (1990) Biochemistry 29, 5405-5412. Cells are incubated with an agent in a buffer—such as phosphate buffered saline—at room temperature for about 3 hr. Cell associated and supernatant fractions are separated by centrifugation at 1000 g for 3 min. Proteins are extracted from the cell pellet using TBS with 0.5% each of deoxycholate and NP-40. This extract and the supernatant fraction are then precipitated with 4-10 volumes of ethanol at −20° C. and subjected to SDS-PAGE in 12% acrylamide gels and immunoblotted with a monoclonal antibody that detects PrPC. If the agent has cleaved PrPC then one or more bands that cross-react with PrPC may be seen on the immunoblot or the molecular mass of PrPC may be lower following cleavage with an agent. If the cell that has been contacted with an agent also contains PrPSc, PrPC and PrPSc may be distinguished by digestion with proteinase K since PrPC is sensitive to proteinase K while PrPSc loses only its amino terminus to give rise to a protease-resistant core.

Various methods may be used for the detection of prion proteins—such as Western blotting (Collinge et al. 1996, Nature 383, 685-690), immunoassay (described in WO 9837210), electronic-property probing (described in WO 9831839) and the cell blot procedure (Bosque and Prusiner (2000) J. Virol. 74, 4377-4386).

For example, for the cell blotting procedure, cells may be transferred to a membrane—such as PVDF membrane—using methods well known in the art and treated with proteinase K and denatured. The prion proteins may be immunostained with an antibody—such as an antibody that specifically binds bovine, murine or human PrPSc—such as 15B3 (Korth et al. (1997) Nature 390, 74-77). Following incubation with a labelled polyclonal antibody—such as horseradish peroxidase-conjugated goat anti-mouse IgG1, prion protein may be visualised by enhanced chemiluminescence.

Agent

As used herein, the term “agent” may be a single entity or it may be a combination of entities.

The agent may be an organic compound. Typically the organic compound will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group,—such as a non-fused polycyclic group. For some applications, the agent comprises at least the one of said cyclic groups linked to another hydrocarbyl group.

The agent may contain halogen compounds—such as fluoro, chloro, bromo or iodo groups.

The agent may contain one or more of alkyl, alkoxy, alkenyl, alkylene and alkenylene groups, which may be unbranched- or branched-chain.

The agent may be an amino acid molecule, a polypeptide—such as an enzyme—or a chemical derivative thereof, or a combination thereof.

Preferably, the agent is an enzyme that cleaves PrPC. More preferably, the agent is a lipase that is capable of cleaving a lipid group from PrPC. More preferably, the agent is a phospholipase that catalyses the hydrolysis of a glycerophospholipd from PrPC. More preferably, the agent is phospholipase C that splits the bond between a phosphorous atom and an oxygen atom at C-1 of a glycerol moiety. Most preferably, the agent is phosphatidylinositol-specific phospholipase C (PIPLC) or a derivative thereof.

Without wishing to be bound by theory, PIPLC cleaves the glycosylphosphatidyl inositol moiety linking PrP to the outer surface of the plasma membrane, thereby releasing PrP from the cell surface.

The agent may be a polynucleotide molecule—which may be a sense or an anti-sense molecule.

The agent may be a natural substance, a biological macromolecule, or an extract made from biological materials—such as bacteria, fungi, or animal (particularly mammalian) cells or tissues, an organic or an inorganic molecule, a synthetic agent, a semi-synthetic agent, a structural or functional mimetic, a peptide—such as β-sheet breaking peptides (Soto et al. (2000) Lancet 355, 192-197), a peptidomimetics, a derivatised agent, a peptide cleaved from a whole protein, or a peptides synthesised synthetically (such as, by way of example, either using a peptide synthesiser or by recombinant techniques or combinations thereof, a recombinant agent, an antibody or fragment thereof, a natural or a non-natural agent, a fusion protein or equivalent thereof and mutants, derivatives or combinations thereof.

The agent may also be an isolated antibody or fragment thereof. The term “antibody” as used herein includes but is not limited to, polyclonal, monoclonal, chimeric, single chain, Fab fragments and fragments produced by a Fab expression library. Such fragments include fragments of whole antibodies which retain their binding activity for a prion protein—such as PrPSc, Fv, F(ab′) and F(ab′)2 fragments—as well as single chain antibodies (scFv), fusion proteins and other synthetic proteins which comprise the antigen-binding site of the antibody. Furthermore, the antibodies and fragments thereof may be neutralising antibodies, i.e. those which inhibit biological activity of the substance polypeptides, are especially preferred for diagnostics and therapeutics.

In a preferred aspect of the present invention, the isolated antibody or fragment thereof is a 6H4 monoclonal antibody.

The 6H4 monoclonal antibody is described in WO 98/37210 and recognises residues 144-152 of murine PrP and thus binds to its helix 1 (Korth et al., 1997 Nature 390, 74-77).

The agent may be designed or obtained from a library of compounds, which may comprise peptides, as well as other compounds,—such as small organic molecules.

The agent of the present invention may be capable of displaying other therapeutic properties.

The agent may be used in combination with one or more other pharmaceutically active agents.

If combinations of active agents are administered—such as PIPLC and 6H4—they may be administered simultaneously, separately or sequentially.

Livestock

The term “livestock”, as used herein refers to any farmed animal. Preferably, livestock are one or more of a pig, sheep, cow or bull. More preferably, livestock are a cow or bull.

Treatment

It is to be appreciated that all references herein to treatment refer to the prevention, suppression, alleviation or curing of prion infection.

The treatment may be of mammals—such as livestock and/or humans.

Derivative

The term “derivative” or “derivatised” means an entity that is formed from another entity to which it is structurally related.

This term includes chemical modification. Illustrative of such chemical modifications would be replacement of hydrogen by a halo group, an alkyl group, an acyl group or an amino group.

Stereo and Geometric Isomers

The agents may exist as stereoisomers and/or geometric isomers—e.g. they may possess one or more asymmetric and/or geometric centres and so may exist in two or more stereoisomeric and/or geometric forms. The present invention contemplates the use of the entire individual stereoisomers and geometric isomers of those agents, and mixtures thereof, provided said forms retain the appropriate functional activity (though not necessarily to the same degree).

Pharamaceutical Salt

The agent may be administered in the form of a pharmaceutically acceptable salt.

Pharmaceutically-acceptable salts are well known to those skilled in the art, and for example include those mentioned by Berge et al, in J. Pharm. Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines—such as diethanolamine, salts.

A pharmaceutically acceptable salt of an agent may be readily prepared by mixing together solutions of an agent and the desired acid or base, as appropriate. The salt may precipitate from solution and be collected by filtration or may be recovered by evaporation of the solvent.

An agent may exist in polymorphic form.

An agent may contain one or more asymmetric carbon atoms and therefore exist in two or more stereoisomeric forms. Where an agent contains an alkenyl or alkenylene group, cis (E) and trans (Z) isomerism may also occur. The present invention includes the individual stereoisomers of an agent and, where appropriate, the individual tautomeric forms thereof, together with mixtures thereof.

Separation of diastereoisomers or cis- and trans-isomers may be achieved by conventional techniques, e.g. by fractional crystallisation, chromatography or H.P.L.C. of a stereoisomeric mixture of an agent or a suitable salt or derivative thereof. An individual enantiomer of an agent may also be prepared from a corresponding optically pure intermediate or by resolution,—such as by H.P.L.C. of the corresponding racemate using a suitable chiral support—or by fractional crystallisation of the diastereoisomeric salts formed by reaction of the corresponding racemate with a suitable optically active acid or base, as appropriate.

The present invention also encompasses all suitable isotopic variations of an agent or a pharmaceutically acceptable salt thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that may be incorporated into an agent and pharmaceutically acceptable salts thereof include isotopes of hydrogen, carbon, nitrogen, oxygen, phosphorus, sulphur, fluorine and chlorine—such as ²H, ³H, ¹³C, ¹⁴C, ¹⁵N, ¹⁷O, ¹⁸O, ³¹P, ³²P, 3S, ¹⁸F and ³⁶Cl, respectively. Certain isotopic variations of an agent and pharmaceutically acceptable salts thereof, for example, those in which a radioactive isotope—such as ³H or ¹⁴C is incorporated are useful in drug and/or substrate tissue distribution studies. Tritiated, i.e., ³H, and carbon-14, i.e., ¹⁴C, isotopes are particularly preferred for their ease of preparation and detectability. Further, substitution with isotopes—such as deuterium, i.e., ²H, may afford certain therapeutic advantages resulting from greater metabolic stability, for example, increased in vivo half-life or reduced dosage requirements and hence may be preferred in some circumstances. Isotopic variations of an agent of the present invention and pharmaceutically acceptable salts thereof of this invention can generally be prepared by conventional procedures using appropriate isotopic variations of suitable reagents.

It will be appreciated by those skilled in the art that an agent may be derived from a prodrug. Examples of prodrugs include entities that have certain protected group(s) and which may not possess pharmacological activity as such, but may, in certain instances, be administered (such as orally or parenterally) and thereafter metabolised in the body to form an agent of the present invention which are pharmacologically active.

It will be further appreciated that certain moieties known as “pro-moieties”, for example as described in “Design of Prodrugs” by H. Bundgaard, Elsevier, 1985 (the disclosured of which is hereby incorporated by reference), may be placed on appropriate functionalities of agents. Such prodrugs are also included within the scope of the invention.

The present invention also includes the use of zwitterionic forms of an agent of the present invention.

Solvates

The present invention also includes the use of solvate forms of an agent of the present invention.

Pro-Drug

As indicated, the present invention may also include the use of pro-drug forms of an agent.

Pharmaceutically Active Salt

An agent may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Mimetic

As used herein, the term “mimetic” relates to any chemical, which includes, but is not limited to, a peptide, polypeptide, antibody or other organic chemical, which has the same qualitative activity or effect as a reference agent.

Pharmaceutical Compositions

Pharmaceutical compositions useful in the present invention may comprise a therapeutically effective amount of one or more agents and pharmaceutically acceptable carrier, diluent or excipient (including combinations thereof).

Pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent may be selected with regard to the intended route of administration and standard pharmaceutical practice. Pharmaceutical compositions may comprise as—or in addition to—the carrier, excipient or diluent any suitable binder(s), lubricant(s), suspending agent(s), coating agent(s) or solubilising agent(s).

Preservatives, stabilisers, dyes and even flavouring agents may be provided in pharmaceutical compositions. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Agents may also be used in combination with a cyclodextrin. Cyclodextrins are known to form inclusion and non-inclusion complexes with drug molecules. Formation of a drugcyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If an agent is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

Administration

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Nonviral delivery mechanisms include lipid mediated transfection, liposomes, immunoliposomes, lipofectin, cationic facial amphiphiles (CFAs) and combinations thereof.

The components useful in the present invention may be administered alone but will generally be administered as a pharmaceutical composition—e.g. when the components are in admixture with a suitable pharmaceutical excipient, diluent or carrier selected with regard to the intended route of administration and standard pharmaceutical practice.

For example, the components may be administered (e.g. orally) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients—such as microcrystalline cellulose, lactose, sodium citrate, calcium carbonate, dibasic calcium phosphate and glycine—disintegrants—such as starch (preferably corn, potato or tapioca starch), sodium starch glycollate, croscarmellose sodium and certain complex silicates—and granulation binders—such as polyvinylpyrrolidone, hydroxypropylmethylcellulose (HPMC), hydroxypropylcellulose (HPC), sucrose, gelatin and acacia. Additionally, lubricating agents—such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents—such as water, ethanol, propylene glycol and glycerin—and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial—such as the brain, intratracheal, intravaginal, intracerebroventricular, intracerebral, subcutaneous, ophthalmic (including intravitreal or intracameral), transdermal, rectal, buccal, vaginal, epidural, sublingual.

It is to be understood that not all of the components of the pharmaceutical need be administered by the same route. Likewise, if the composition comprises more than one active component, then those components may be administered by different routes.

If a component is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) useful in the present invention may be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane—such as 1,1,1,2-tetrafluoroethane (HFA 134A™) or 1,1,1,2,3,3,3-heptafluoropropane (HFA 227EA™)—carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base—such as lactose or starch.

Alternatively, the component(s) may be administered in the form of a suppository or pessary, or it may be applied topically in the form of a gel, hydrogel, lotion, solution, cream, ointment or dusting powder. The component(s) may also be dermally or transdermally administered, for example, by the use of a skin patch. They may also be administered by the pulmonary or rectal routes. They may also be administered by the ocular route. For ophthalmic use, the compounds may be formulated as micronised suspensions in isotonic, pH adjusted, sterile saline, or, preferably, as solutions in isotonic, pH adjusted, sterile saline, optionally in combination with a preservative—such as a benzylalkonium chloride. Alternatively, they may be formulated in an ointment—such as petrolatum.

For application topically to the skin, the component(s) may be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it may be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Daily or frequent administration of an agent may be required if clearance is rapid and/or penetration of the blood-brain barrier is slow.

In a preferred aspect, the present invention provides a method of treating or preventing prion infection in a subject comprising administering to said subject a 6H4 monoclonal antibody wherein said 6H4 monoclonal antibody is administered as an encapsulated hybridoma.

The 6H4 monoclonal antibody may be used as an immune modulator—such as a vaccine that is used for inoculation against prion infection. Preferably, the immune modulator is used for passive immunisation, which has its usual meaning in the art and involves the introduction of pre-formed antibodies to a particular antigen—such as PrPC and/or PrPSc.

Administration of 6H4 as an encapsulated hybridoma may reduce clearance of the antibody and/or improve penetration of the blood-brain barrier. Preferably, the encapsulated hybridoma maintains detectable levels of anti-PrP antibodies for several weeks.

Preferably, encapsulated hybridomas cells are administered by intracerebral, intraperitoneal or intrathecal insertion.

The general methodology for making monoclonal antibodies by hybridomas is well known. Antibody-producing cell lines may be created by cell fusion, and also by other techniques—such as direct transformation of B lymphocytes with oncogenic DNA, or transfection with Epstein-Barr virus. Panels of monoclonal antibodies produced against orbit epitopes may be screened for various properties; i.e. for isotype and epitope affinity.

Preferably, the hybridoma is a cell line capable of producing the monoclonal antibody 6H4 deposited under DSM.ACC2295 (WO 98/37210).

Matrices used to encapsulate cells and organisms include matrices based on alginate gel technology. For example, U.S. Pat. Nos. 4,401,456 and 4,400,391 disclose processes for preparing alginate gel beads containing bioactive materials. The most usual hydroxyl polymers used for encapsulating biomaterials are alginate, polyacrylamide, carrageenan, agar, or agarose. Alginate and carrageenan may be manufactured in spherical form with encapsulated material. by ionotropic gelling, i.e., the alginate is dropped down into a calcium solution and the carrageenan into a potassium solution. The resulting beads are stable only in the presence of ions (calcium and potassium, respectively). The use of ultrasonic nozzles has offered a new way of making smaller microspheres with very good control over the size of the droplets (Ghebre-Sellassie (1989) “Pharmaceutical pelletilization technology,” In J. Swarbrick (ed.) Drugs and the pharmaceutical sciences: Vol. 37. Pharmaceutical pelletilization technology, New York: Marcel Dekker). Liquid is supplied at low pressure and droplets are formed at the tip of the nozzle by ultrasonic frequency.

Cellulose acetate phthalate (CAP) is a polyelectrolyte containing ionizable carboxyl groups. It is an enteric coating widely used in the industry for coating tablets. Enteric coatings protect the drug from the gastric juices (pH range 1-6) (Yacobi & Walega (1988) Oral sustained release formulations: Dosing and evaluation, Pergammon Press). CAP serves this purpose by being virtually insoluble below pH 6.0. Aquateric is a commercially available pseudolatex containing CAP. Other constituents include Pluronic F-68, Myvacet 9-40, polysorbate 60 and <4% free phthalic acid (McGinity [1989], supra). Both CAP and aquateric can be fabricated into microspheres by first dissolving them in pH 7.0 distilled deionized water and dropped in acidic solution. Others have used coacervation as the method for microencapsulation (Merkle & Speiser (1973) J Pharmac. Sci. 62:1444-1448).

The use of various matrices to encapsulate cells and organisms for implantation in the body has been previously reported (Sun, A. M. (1988) “Microencapsulation of pancreatic islet cells: A bioartificial endocrine pancreas,” In Mosbach, K. (ed.) Methods in enzymology. Vol. 137, Academic Press, Inc.). Pancreatic cells have been utilized in vitro and in vivo for the production and delivery of insulin. Long term in vivo (in rats) studies of alginate microcapsules containing islet cells, implanted in the peritoneal cavity, have shown great biocompatibility with no cell adhesion to the capsules and a reversal to normal of the previously diagnosed diabetic rats (Sun, A. M., Z. Cai, Z. Shi, F. Ma, G. M. O'Shea [1987] Biomaterials, Artificial Cells, and Artificial Organs 15(2):483-496).

In a preferred embodiment, encapsulated hybridomas cells are prepared by loading cells into preformed capsules—such as polyethersulfone microporus hollow fibers which are available in a wide range of controlled pore sizes. Encapsulated hybridomas cells may also be prepared by embedding cells in alginate beads. Alginate solutions containing dispersed cells may be gelled by adding into calcium solutions, which results in small beads, 300-500 μm diameter, with cells entrapped in the meshes. Such beads retain viable cells (70-80%) for many months, both in vitro and in vivo, and the cell products are discharged into the medium. This approach may be used to deliver a variety of proteins to various sites—such as the brain.

Many variations have been reported for preparing alginate beads—such as the addition of polylysine, PLL (a polyelectrolyte), coating with PLL and alginate, use of Ba⁺⁺ rather than Ca⁺⁺ to increase mechanical stability. Immunoglobulins, in particular IgG may diffuse out of the bead in vitro and in vivo. Antibodies against alginate have been reported of which a high proportion of guluronic acid in the alginate decreased immunogenicity. Also antibodies against cells encapsulated in alginate have been observed, due to leaching of cellular proteins. However, cells in the beads are not affected, because they are isolated from cytotoxic T cells and protected from complement-mediated lysis.

For some embodiments, preferably, the immune modulator comprises or is based on a humanised 6H4 monoclonal antibody.

Humanised antibodies may be obtained using various methods well known in the art (for example as described in U.S. Pat. No. 239,400). Monoclonal antibodies may be obtained by immunising immunologically humanised mice with for example, a recombinant substantially purified preparation of PrP. Immunologically humanised mice are commercially available through Abgenix or Medarex for example.

The agent may be administered in combination with an adjuvant to provide a generalised stimulation of the immune system.

The encapsulated hybridoma may be administred before or after prion infection has been determined in a subject.

Dose Levels

Typically, a physician will determine the actual dosage, which will be most suitable for an individual subject. The specific dose level and frequency of dosage for any particular patient may be varied and will depend upon a variety of factors including the activity of the specific compound employed, the metabolic stability and length of action of that compound, the age, body weight, general health, diet, mode and time of administration, rate of excretion, drug combination, the severity of the particular condition, and the individual undergoing therapy.

PIPLC administered before and/or during exposure of a susceptible cell to prions in quantities of 0.25, 0.5, 1 and 2 units/ml prevents appearance of PrPSc.

6H4 administered before and/or during exposure of a susceptible cell to prions in quantities of 2.5, 5, 10 and 20 μg/ml prevents appearance of PrPSc.

PIPLC administered in quantities of 0.25 or 0.5 units/ml causes rapid loss of PrPSc when administered to susceptible cells chronically infected with prions.

6H4 administered in quantities of 3 μg/ml causes rapid loss of PrPSc when administered to susceptible cells chronically infected with prions.

2.5, 5, 10 and 20 μg/ml of 6H4 administered to cells chronically infected with prions remain devoid of PrPSc for 2, 4 or 6 weeks after removal of the agent.

Thus, in a preferred aspect, 0.25 units/ml or greater of PIPLC and/or 2.5 μg/ml or greater of 6H4 are administered for the treatment or prevention of prion infection in a subject.

Formulation

The component(s) may be formulated into a pharmaceutical composition,—such as by mixing with one or more of a suitable carrier, diluent or excipient—by using techniques that are known in the art.

### FIGURES

The present invention will now be described by way of example, in which reference is made to the following Figures:

FIG. 1 represents the susceptibility to scrapie infection and PrP level of various sublines of N2a cells. N2a populations as propagated routinely in the lab and single clones transformed with a PrP expression or a control vector are seeded into 24-well plates (2×10⁴ cells/well) and grown to confluence. (a) Cultures are exposed for 3 days to purified mouse PrPSc (RML strain, 20 ng/ml), cultured for 29 days (8 passages) and assayed for PrPSc formation by the cell blot assay. (b) Prion-susceptible N2a/Bos2 and resistant N2a/2M11 cells are exposed for 3 days to the dilutions indicated of infected 10% brain homogenate, cultured for 14 days (3 passages) and assayed for PrPSc formation. Cells exposed to a 10⁻⁴ dilution are still slightly positive (c) Western blot analysis of N2a sublines is performed using monoclonal anti-PrP antibody 6H4. Cells transfected with the expression plasmid for mouse PrP^{c}, MHM2 PrP or MH2M PrP are indicated by mo, M2 or 2M, respectively. BOS designates cells cotransfected with pSVneo and pEF-BOS-EX. N2a, the original uncloned cells, as well as the highly susceptible N2a/Bos2 cell line show similar, low expression of PrPC as compared to the non-susceptible moS or 2M11 lines.

FIG. 2 represents anti PrP antibody 6H4 and PIPLC preventing infection of N2a/Bos2 cell with scrapie prions and abrogate PrPSc accumulation in chronically infected cells. (a) N2a/Bos2 cells are incubated for 2 h with antibody 6H4 or PIPLC at the concentrations indicated and exposed to 0.1% scrapie-infected brain homogenate (fmal concentration) for 3 days. After culturing for 14 days (3 passages) in the absence of PIPLC or in the continued presence of 6H4, PrPSc expression is determined. (b,c) Chronically scrapie-infected N2a/Bos2 cells are cultured for (b) 3 days at the levels of antibody 6H4 or of PIPLC indicated or (c) 14 days (3 passages) at the concentrations of antibody 6H4 indicated, and PrPSc accumulation is monitored. (d) Chronically scrapie-infected N2a/Bos2 cells are exposed to 6H4 at the concentrations indicated for 2 weeks and further cultured in the absence of the antibody for 6 weeks. Cultures are split 1:5 every 34 days. There is no reappearance of PrPSc. “Cell staining” refers to staining of the membranes with ethidium bromide to monitor efficiency of transfer of the cell layer. IN, chronically scrapie-infected N2a/Bos2 cells.

FIG. 3 represents chronically infected N2a/Bos2 cells “cured” of PrPSc by antibody 6H4 treatment continuing to produce PrP and their susceptibility to reinfection. (a) Chronically infected N2a/Bos2 cells, treated with antibody 6H4 for 2 weeks at the concentrations indicated and propagated in its absence for 66 days were not exposed (top 2 rows) or exposed to 0.1% RML-infected mouse brain homogenate for 3 days (bottom 2 rows). After culturing for 14 days, PrPSc is monitored by the cell blot assay. (b) Relative susceptibility to prions of N2a/Bos2 cells (BOS2) and N2a/Bos2 cells “cured” by exposure to antibody 6H4 at 20 μg/ml is determined by exposing cultures to various dilutions of RML-infected mouse brain homogenate for 3 days and determining PrPSc as above. (c) Levels of PrPC and PrPSc in various sublines are determined by Western blotting. Chronically infected N2a/Bos2 cells, treated for 2 weeks with antibody 6H4 at the concentrations indicated are passaged for 84 days after antibody withdrawal. Cells are lysed and samples corresponding to 2.25×10⁵ cells are incubated in the presence (+PK) or absence (−PK) of proteinase K (5 μg/ml) for 90 minutes at 37° C. Western blotting is performed as described in Methods. UN, uninfected N2a/Bos2 cells and I-BOS2, chronically prion-infected BOS2 cells. Molecular weight markers are indicated at the left of each panel.

FIG. 4 represents a model to explain abolition of PrPSc by anti PrP antibody (or PIPLC). PrPC is attached to the membrane by a glycosylphosphatidyl inositol anchor and cycles between the cell surface and an endocytic compartment (43). In scrapie-infected cells, PrPC is recruited into PrPSc “seeds” (44), which may be located at the cell surface and/or in the endocytic/lysosomal compartment. PrPSc is degraded with a half-life of about 15 h (37); if PrP^{C} is prevented from converting to PrPSc by either a blocking antibody or by being stripped from the cell surface by PIPLC, PrPSc will diminish and ultimately disappear.

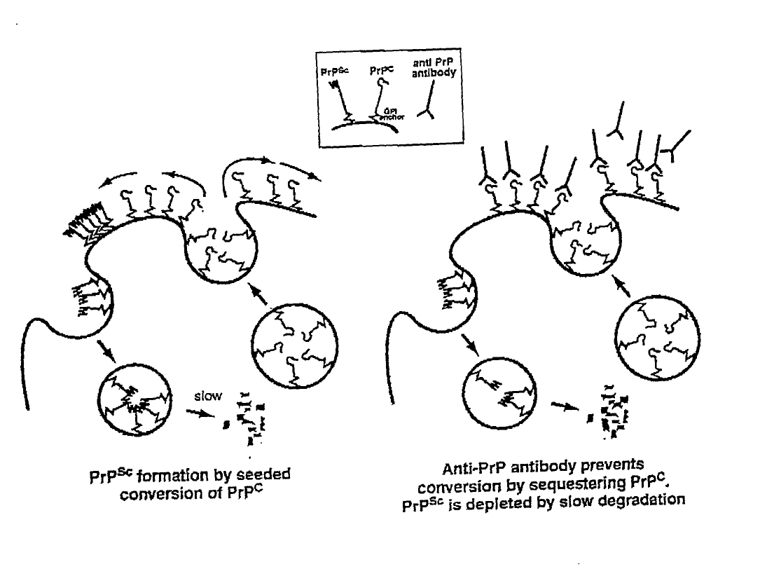

### EXAMPLES

Materials and Methods

Cell Culture.

Mouse neuroblastoma N2a cells are cultured in OPTI-MEM supplemented with 10% fetal calf serum (FCS) and penicillin G-streptomycin (complete medium) at 37° C. in 5% CO₂. Cells are routinely split 1:5 every 3 to 4 days and grown in 10-cm dishes (Corning Costar, New York, N.Y., USA). PIPLC was from Sigma (St.Louis, Mo., USA). Antibodies used are of the monoclonal IgG1 subtype: anti PrP (6H4, Prionics AG), anti APP (22C11, Boehringer Mannheim, Germany) and anti β-actin (AC-15, Sigma, St.Louis Mo., USA).

Prion Infection of N2a Cells.

Brains from scrapie-sick mice infected with Rocky Mountain Laboratory (RML) mouse-adapted prions are homogenized by passing 8 times each through 21G and 25G needles and adjusted to 10% (w/v) with 1×Dulbecco's phosphate-buffer saline (D-PBS; Gibco BRL, Glasgow, UK). After centrifuiging 5 min at 1,000 rpm (Eppendorf Centrifuge 5415c, Hamburg, Germany) and room temperature, supernatants are recovered and stored at −80° C. Mouse PrPSc is partially purified as described by Bolton et al. (1987) Arch. Biochem. Biophys. 258, 579-590. Cells (2-5×10⁴ in 1 ml medium) are seeded into 24-wells plates (Corning Costar) and cultured for 1-2 days before exposure to either 20 ng/ml of purified PrPSc (RML strain) or RML-infected mouse brain homogenate, diluted as indicated with complete medium. The inoculum is removed after 3 days and the cells are split 1:5 every 3-4 days. After 14 days the cells are assayed for PrPSc by the cell blot procedure (Bosque, P. J. & Prusiner, S. B. (2000) J. Virol. 74, 4377-4386).

Selection of Prion-susceptible and Prion-resistant N2a Sublines.

N2a cells are cotransfected with pSVneo (Southern, P. J. & Berg, P. (1982) J. Mol. Appl. Genet. 1, 327-341) that confers neomycin resistance and a plasmid (pEF-Bos-EX; Murai et al. (1998) Proc. Natl. Acad. Sci. USA 95, 3461-3466) containing a “half-genomic” PrP transcription unit (Fischer et al. (1996) EMBO J. 15, 1255-1264) with the open reading frame of wild type PrP, MHM2 PrP (Scott et al. (1993) Cell 73, 979-988; Scott et al. (1992) Protein Sci. 1, 986-997) or MH2M PrP (Scott et al. (1993) Cell 73, 979-988; Scott et al. (1992) Protein Sci. 1, 986-997) under the control of the EF1α promoter. The aim is to generate N2a cell lines that overexpress mouse or hamster-mouse hybrid PrP molecules and thereby facilitate infection with mouse and/or hamster prions (Scott et al. (1992) Protein Sci. 1, 986-997). As a negative control, cotransformation is performed with pSVneo and pEF-BOS-EX (devoid of an open reading frame). After selection in G418 at 0.7 mg/ml, clones of each group are tested for susceptibility to infection by RML scrapie prions, as described above. Clone N2a/Bos2 is selected as the most susceptible subline and aliquots thereof and of N2a/Bos2 cells chronically infected with RML prions are kept at −80° C.

Cell Blot Assay for PrPSc.

The assay is performed as described (Bosque, P. J. & Prusiner, S. B. (2000) J. Virol. 74, 4377-4386). In short, cells are transferred to a PVDF membrane, treated with proteinase K, denatured, immunostained with antibody 6H4 followed by horseradish peroxidase-conjugated goat anti-mouse IgG1, and visualized by enhanced chemiluminescence (ECL kit; Pierce, Rockford Ill., USA). After exposure, the membrane is stained for 15 min with 0.5 μg/ml ethidium bromide and photographed in UV light, to document the transfer of the cell layer.

Western Blot Analysis.

Samples (15 μg total protein) are run through 16% SDS-polyacrylamide gels (8×8 cm) and transferred to a PVDF membrane by electroblotting at 35 V for 1 h. PrP is detected by incubation with 200 ng/ml monoclonal anti-PrP antibody 6H4 (Korth et al. (1997) Nature 390, 74-77) (Prionics A G, Zürich, Switzerland) followed by horseradish peroxidase-conjugated goat anti-mouse IgG1 (Zymed, South San Francisco, Calif., USA; 10,000-fold dilution in TBST containing 1% dry milk) at room temperature for 1 h. The immune complex is visualized by enhanced chemiluminescence (ECL kit, Pierce, Rockford, Ill., USA).

### Example 1

Isolation of Cell Lines Susceptible and Resistant to RML Scrapie Prions.

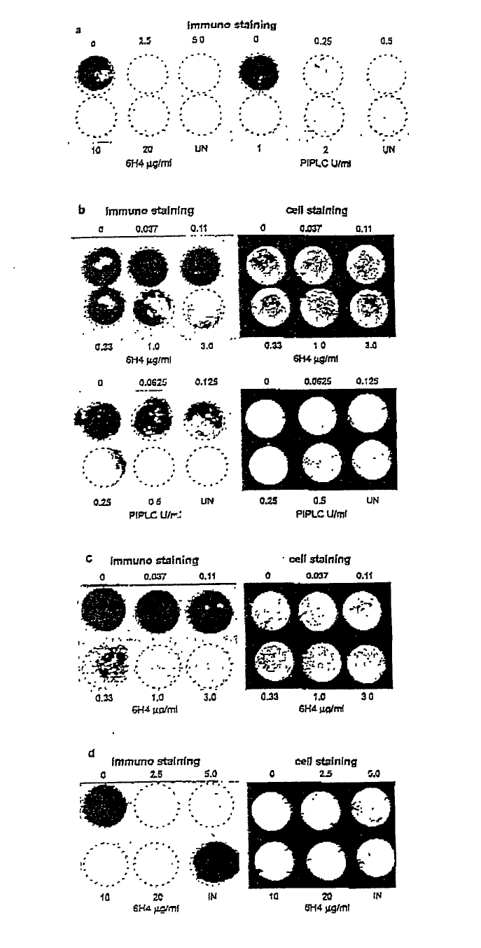

N2a cells are transformed with PrP expression (or control) plasmids with the intent of raising their PrP level and thereby rendering them more susceptible to infection with prions from various sources. Transformed clones are assayed for their susceptibility to RML prion infection by an immuno-blotting assay (Bosque, P. J. & Prusiner, S. B. (2000) J. Virol. 74, 4377-4386) (FIG. 1a). Unexpectedly, the most susceptible line, N2a/Bos2, was derived from cells that had been transformed with the control plasmid. In order to assess the proportion of cells susceptible to infection, N2a/Bos2 cells were subcloned and assayed by the blotting procedure; 49% of the cells were susceptible to infection (data not shown).

N2a/Bos2 cells and a prion-resistant line, N2a/2M11, (transformed with a PrP expression plasmid) are challenged with mouse-scrapie-infected brain extract at various dilutions and passaged for 2 weeks. N2a/Bos2 cultures become PrPSc-positive after exposure to scrapie-infected 10% brain homogenate diluted to 10⁻⁴, while the resistant line remains negative at all dilutions tested (FIG. 1b).

The susceptible cell line N2a/Bos2 expresses PrP at about the same low level as the original N2a cells, while the resistant line expresses PrP at a level 10 or more times higher, as shown by Western blot analysis (FIG. 1c). PrP is expressed at the cell surface of all cell lines, as evidenced by biotinylating intact cells, immunoprecipitating the extracted proteins with anti PrP antibody and subjecting them to Western blot assay with horseradish peroxidase-labelled streptavidin (data not shown).

Thus, cell lines susceptible and resistant to RML scrapie prions are prepared.

### Example 2

PIPLC Prevents Infection of the Susceptible N2a Cell Line by Scrapie Prions.

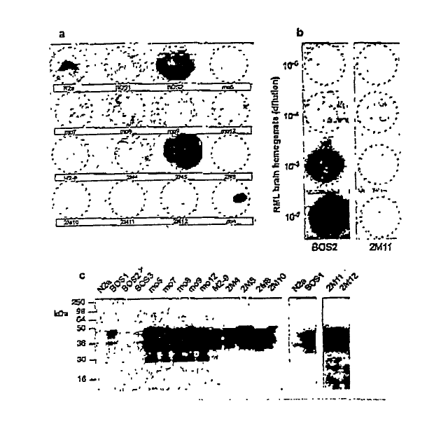

PIPLC is added to a medium containing susceptible N2a cells at various concentrations 2 h before and during exposure of N2a/Bos2 cells to 0.1% scrapie-infected brain homogenate. After 3 days, the cells are washed and further cultured for 2 weeks, splitting 1:5 every 3-4 days. As assayed by the blotting procedure after 14 days, PIPLC at 0.25 units/ml sufficed to prevent appearance of PrPSc (FIG. 2a).

Thus, it is demonstrated that PIPLC prevents the infection of susceptible N2a cells by scrapie prions when the cells are contacted with PIPLC either before or during exposure to prions.

### Example 3

Anti PrP Monoclonal Antibody 6H4 Prevents Infection of the Susceptible N2a Cell Line by Scrapie Prions.

Antibody 6H4 is added to the medium at various concentrations 2 h before and during exposure of N2a/Bos2 cells to 0.1% scrapie-infected brain homogenate. After 3 days, the cells are washed and further cultured for 2 weeks, splitting 1:5 every 3-4 days. During this period antibody 6H4 treatment is continued. As assayed by the blotting procedure after 14 days, antibody 6H4 at 2.5 μg/ml prevents appearance of PrPSc (FIG. 2a). Two other IgG1 monoclonal antibodies, anti APP and anti β-actin, at concentrations up to 5 μg/ml are used as controls (data not shown).

Accordingly, it is demonstrated that 6H4 prevents the infection of susceptible N2a cells by scrapie prions when the cells are contacted with 6H4 either before or during exposure to prions.

### Example 4

Rapid Loss of PrPSc Elicited by PIPLC.

Chronically prion-infected N2a/Bos2 cells are exposed to various concentrations of PIPLC and cultured on coverslips without passaging. After 3 days, PrPSc is barely detectable using PIPLC at 0.25 units/ml (FIG. 2b).

Therefore, it is shown that PIPLC can cure chronically prion-infected N2a/Bos2 cells of infection by scrapie prions.

### Example 5

Rapid loss of PrPSc elicited by anti PrP monoclonal antibody 6H4.

Chronically prion-infected N2a/Bos2 cells are exposed to various concentrations of antibody 6H4 and cultured on coverslips without passaging. After 3 days, PrPSc is barely detectable with 6H4 at 3 μg/ml (FIG. 2b). After culturing for 2 weeks, splitting 1:5 every 3-4 days, as little as 1 μg/ml 6H4 leads to disappearance of PrPSc (FIG. 2c).

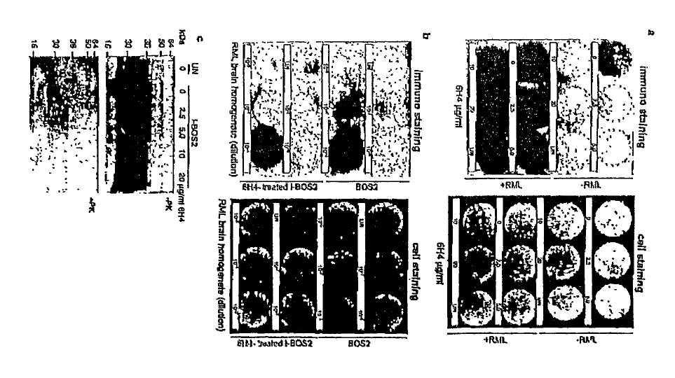

To determine whether the depletion of PrPSc is transient or permanent, chronically infected N2a/Bos2 cells are cultured for 2 weeks with various concentrations of antibody 6H4 and subsequently in its absence. Samples assayed 2, 4 (data not shown) or 6 weeks (FIG. 2d) after removal of the antibody are devoid of PrPSc, in contrast to the chronically infected, untreated cells. “Cured” cells are still susceptible to scrapie prion infection (FIG. 3a,b) and express PrP^{C} at the original level (FIG. 3c).

Therefore, it is shown that 6H4 can cure chronically prion-infected N2a/Bos2 cells of infection by scrapie prions.

### Example 6

Humanised Anti-PrP Antibodies

“Human” monoclonal antibodies are obtained by immunising “immunologically humanised” mice (available from Abgenix or Medarex) with PrPSc and producing the selected antibody in hybridomas.

### Example 7

Encapsulation of Hybridoma Cells

Hybridoma cells are loaded into preformed capsules (polyethersulfone microporus hollow fibers).

### Example 8

Administration of Encapsulated Hybridoma Cells Intracerebrally or Intraperitoneally

25 alginate beads containing or not containing hybridoma cells are injected in a final volume of 25 μl, intracereberally into 4 mice each.

100 and 500 beads, respectively, are injected that contain anti-PrP producing hybridoma cells are injected intraperitoneally into 4 mice each.

Antibody levels are determined after 1 and 4 weeks.

### Example 9

Intracerebral Inoculation; Encapsulated Hybridoma Cells Administered Intracerebrally or Intraperitoneally

5 or 25 beads (suspended in 20 μl PBS; a bead of 500 μm diameter has a volume of about 0.06 μl) containing anti-PrP hybridoma cells or mock hybridoma cells are injected stereotaxically into the brain of tga20 mice (a) simultaneously with RML inoculation (b) 35 days after inoculation. Inoculation is intracerebrally with 1% RML-infected brain homogenate in a volume of 10 μl.

The control used is mice inoculated intracerebrally with RML, without or (simultaneously) with intracerebrally or intraperitoneally administered “mock” beads.

The monoclonal antibody in serum during experiment and the survival time of the mice is monitored.

Histopathology, brain and spleen infectivity, Western blots of brain and spleen and the presence of the monoclonal antibody in brain, spleen and serum after 60 days and at the terminal stage or after 200 days is determined.

### Example 10

Intraperitoneal Administration of Encapsulated Hybridoma Cells

100 or 500 beads (in 100 μl PBS) are injected intraperitoneally into tga20 mice (a) simultaneously with intraperitoneal scrapie inoculation (b) 35 days after inoculation. Inoculation is with 100 μl 1% RML-infected brain homogenate, intraperitoneally.

Mice inoculated with RML intraperitoneally without or (simultaneously) with intraperitoneally administered “mock” beads are used as the control.

During the experiment, the monoclonal antibody in the serum is determined. Additionally, the survival time, histopathology, brain and spleen infectivity, monoclonal antibody in brain, spleen and serum at time of death are also determined. Two mice of each set to be sacrificed 60 days after infection are examined.

All publications mentioned in the specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in molecular biology or related fields are intended to be within the scope of the following claims.

### PRION REFERNCES

Aguzzi, A.; Brandner, S. Lancet 354: 22-25, 1999.

Aguzzi, A.; Weissmann, C. A suspicious signature. Nature 383: 666-667, 1996.

Basler, K. et al., Cell 46: 417-428, 1986.

Beyreuther, K.; Masters, C. L. Nature 370: 419-420, 1994.

Bueler, H. et al., Cell 73: 1339-1347, 1993.

Bueler, H. et al., Molec. Med. 1: 19-30, 1994.

Carlson, G. A. et al., Cell 46: 503-511, 1986.

Chapman, J. et al., Neurology 46: 758-761, 1996.

Chiesa et al. Neuron 21: 1339-1351, 1998.

Collinge, J. Hum. Mol. Genet. 6: 1699-1705, 1997.

Collinge, J. et al., Lancet 336: 7-9, 1990.

Collinge, J et al. Lancet 337: 1441-1442, 1991.

Collinge, J. et al., Nature 383: 685-690, 1996.

Collinge, J. et al., Nature 370: 295-297, 1994.

Duchen, L. W et al. Brain 116: 555-567, 1993.

Farlow, M. R. Neurology 39: 1446-1452, 1989.

Forloni, G.; et al., Nature 362: 543-546, 1993.

Gajdusek, D. C. Europ. J. Epidemiol. 7: 567-577, 1991.

Ghetti, B et al., Neurology 39: 1453-1461, 1989.

Griffith, J. S.: Self-replication and scrapie. Nature 215: 1043-1044, 1967.

Hegde, R. S. et al., Science 279:827-834, 1998.

Hegde, R. S. et al.,. Nature 402: 732-736, 1999.

Horwich & Weissman, Cell 89: 499-510, 1997.

Hsiao, K. et al., Nature 338: 342-345, 1989.

Hsiao, K et al., Neurobiol. Aging 11: 302, 1990.

Hsiao, K et al.,. Nature Genet. 1: 68-71, 1992.

Hsiao, K. K et al., Proc. Nat. Acad. Sci. 91: 9126-9130, 1994.

Jendroska, K etal., J. Neurol. Neurosurg. Psychiat. 57: 1249-1251, 1994.

Kocisko, D. A et al., Nature 370: 471-474, 1994.

Krasemann, S et al., Molec. Brain Res. 34: 173-176, 1995.

Kuwahara, C. et al., Nature 400: 225-226, 1999.

Liao, Y.-C. J. et al., Science 233: 364-367, 1986.

Lindquist, S. Cell 89: 495-498, 1997.

Mallucci, G. R et al., Brain 122: 1823-1837, 1999.

Manson, J. C et al., Neurodegeneration 3: 331-340, 1994.

Manuelidis, L et al., EMBO J. 6: 341-347, 1987.

Mestel, R. Science 273: 184-189, 1996.

Montrasio F et al. Science 2000;288: 1257-9.

Oesch, B et al.,. Cell 40: 735-746, 1985.

Owen, F et al., Molec. Brain Res. 13: 155-157, 1992.

Pablos-Mendez, et al., Lancet 341: 159-161, 1993.

Palmer, M. S.; Collinge, J. Hum. Mutat. 2: 168-173, 1993.

Perry, R. T. et al., Am. J. Med. Genet. 60: 12-18, 1995.

Prusiner, S. B. Annu. Rev. Med. 38: 381-398, 1987.

Prusiner, S. B. Ann. Rev. Microbiol. 48: 655-686, 1994.

Prusiner, S. B. Science 216: 136-144, 1982.

Prusiner, S. B. Cold Spring Harbor Symp. Quant. Biol. 61: 473-493, 1996.

Puckett, C et al.,. Am. J. Hum. Genet. 49: 320-329,1991.

Riek, R et al., Proc.Nat. Acad. Sci. 95: 11667-11672, 1998.

Rivera, H et al.,. J. Med. Genet. 26: 626-630, 1989.

Robakis, N. K et al., Biochem. Biophys. Res. Commun. 140: 758-765, 1986.

Sakaguchi, S et al., Nature 380: 528-531, 1996.

Schellenberg, G. D. et al., Am. J. Hum. Genet. 49: 511-517, 1991.

Schnittger, S et al., Genomics 14: 740-744, 1992.

Scott, M et al., Cell 59: 847-857, 1989.

Shmerling, D et al., Cell 93: 203-214, 1998.

Sparkes, R. S et al., Proc. Nat. Acad. Sci. 83: 7358-7362, 1986.

Supattapone, S et al., Cell 96: 869-878, 1999.

Tagliavini, F etal., EMBO J. 10: 513-519, 1991.

Telling, G. C et al., Science 274: 2079-1962, 1996.

Ter-Avanesyan, M. D. et al., Genetics 137: 671-676, 1994.

Tobler, I et al., Nature 380: 639-642, 1996.

Wickner, R. B. Science 264: 566-569, 1994.

Windl, O et al., Hum. Genet. 105: 244-252, 1999.

1. A method of treating or preventing prion infection in a subject comprising administering to said subject a therapeutically effective amount of an agent wherein said agent cleaves PrPC.

2. A method according to claim 1 wherein said agent is phosphatidylinositol-specific phospholipase or a derivative thereof.

3. A method according to any one of the preceding claims wherein said prion infection is caused by a mammalian prion protein.

4. A method according to claim 3 wherein said prion infection is caused by a livestock or a human prion protein.

5. A pharmaceutical composition comprising an agent and a pharmaceutically acceptable carrier, diluent, excipient or adjuvant or any combination thereof wherein said agent agent cleaves PrPC.

6. A pharmaceutical composition according to claim 5 wherein said agent is phosphatidylinositol-specific phospholipase or a derivative thereof.

7. A method of treating or preventing prion infection in a subject comprising administering to said subject a therapeutically effective amount of 6H4 monoclonal antibody wherein said 6H4 monoclonal antibody is administered as an encapsulated hybridoma.

8. A method according to claim 7 wherein the 6H4 monoclonal antibody is a humanised antibody.
